(19) United States
(12) Patent Application Publication
(10) Pub. No.: US 2025/0387620 A1
(43) Pub. Date: Dec. 25, 2025

(54) DATA STORAGE AND MAINTENANCE IN IMPLANTABLE DEVICES

(71) Applicant: Boston Scientific Neuromodulation Corporation, Valencia, CA (US)

(72) Inventors: Rosana Esteller, Santa Clarita, CA (US); Philip Leonard Weiss, Sherman Oaks, CA (US)

(21) Appl. No.: 19/232,629

(22) Filed: Jun. 9, 2025

Related U.S. Application Data
(60) Provisional application No. 63/663,606, filed on Jun. 24, 2024.

(51) Int. Cl. A61N 1/08; A61N 1/36; G16H 10/60; G16H 20/40

(52) U.S. Cl. CPC A61N 1/08; A61N 1/36132; A61N 1/36139; G16H 10/60; G16H 20/40

(57) ABSTRACT

Systems and methods for storing and managing physiological data in implantable medical devices (IMDs) are disclosed. An ambulatory medical-device system includes a sensor circuit to sense a physiological signal from a patient, a memory circuit, and a controller circuit. The controller circuit determines a feature value margin based on a variability metric of baseline signal feature values from a physiological signal sensed during a baseline condition, determines test signal feature values from a physiological signal sensed during a test condition different from the baseline condition, and selectively stores in the memory circuit a subset, less than an entirety, of the determined test values that fall outside the feature value margin about a reference feature value. The stored test values of the signal feature can be transmitted to an external device for inspection or further processing.

### CLAIM OF PRIORITY

This application claims the benefit of U.S. Provisional Application No. 63/663,606, filed on Jun. 24, 2024, which is hereby incorporated by reference in its entirety.

### TECHNICAL FIELD

This document relates generally to medical devices, and more particularly, but not by way of limitation, to physiological data storage and maintenance in implantable medical devices.

### BACKGROUND

Implantable medical devices (IMDs) have been used for monitoring patient health or disease states and delivering therapies when necessary. For example, implantable neuromodulation (also referred to as “neurostimulation” or “neural stimulation”) devices have been used to manage a number of neurological or other conditions. Neuromodulation is often used to produce excitatory, inhibitory, and/or other effects. Examples of neuromodulation include Spinal Cord Stimulation (SCS), Deep Brain Stimulation (DBS), Peripheral Nerve Stimulation (PNS), and Functional Electrical Stimulation (FES). SCS systems have been used as a therapeutic modality for the treatment of chronic pain syndromes. PNS has been used to treat chronic pain syndrome and incontinence, with a number of other applications under investigation. FES systems have been applied to restore some functionality to paralyzed extremities in spinal cord injury patients. DBS can be used to treat a variety of diseases or disorders.

Some IMDs have sensing circuitry that can sense physiological information from a patient, and a pulse generator to generate therapeutic electrostimulation pulses. The pulse generator may be electrically coupled to one or more leads each including a plurality of stimulation electrodes. The stimulation electrodes are in contact with or near target tissue to be stimulated, such as nerves, muscles, or other tissue. The pulse generator can generate electrostimulation pulses that are delivered to the target tissue via the electrodes in accordance with an electrode configuration or a stimulation setting. A feedback controller can determine or adjust stimulation setting based on the physiological information sensed from the patient. The sensed physiological information may be stored in an internal memory of the IMD. The stored physiological information can be transmitted to an external device for inspection or further processing.

### SUMMARY

Physiological data storage and maintenance are important functionalities of implantable medical devices (IMDs). With the advancement of sensor technologies, modern IMDs have increased capabilities of sensing and storing onboard a large volume of physiological data. For example, implantable neuromodulation devices may include, or be coupled to, ambulatory sensors or sensing electrodes to sense electrocardiography (ECG), electromyography (EMG), electroencephalography (EEG), electrospinogram (ESG), Evoked Resonant Neural Activity (ERNA) (also referred to as deep brain stimulation local evoked potentials (DLEP) or evoked oscillatory neural responses (EONR)), respiration, motion or acceleration in different locations (e.g., finger, spinal cord), transcutaneous impedance, temperature, and pupil dilation, among other types of physiological data. In addition to the raw physiological data, processed data such as features extracted from physiological signals may also be stored in the IMD. In many occasions, the stored raw or processed physiological data may be downloaded to an external device (e.g., a physician's programmer, a data server, or a smart handheld device) for inspection, further processing, or decision making. The present inventors have recognized challenges in physiological data storage and maintenance in IMDs due to constraints in storage capacity, onboard power, communication bandwidth, and transmission energy (for data communication between IMD and an external device). Even with the advancement in battery/memory technologies, it remains desirable to conserve battery power and optimize memory usage, which are important considerations to improve device efficiency and to extend device longevity.

Embodiments of the present subject matter provide systems, device and methods for storing and managing physiological data in a battery-powered ambulatory medical device, such as an IMD. The physiological data may be acquired by implantable, wearable, or other ambulatory sensors included in, or otherwise communicatively coupled to, an ambulatory medical device. The ambulatory medical device can identify, from signal feature values extracted from physiological signal or biomarker data, a subset of feature values that satisfy a specific condition with respect to a feature value margin correlated to variability of “baseline” signal feature values determined from a physiological signal during a baseline patient condition. By storing only the identified subset, less than the entirety, of the signal feature values, device memory usage and battery power consumption can be reduced.

An example (e.g., “Example 1”) of an ambulatory medical-device system includes a sensor circuit configured to sense a physiological signal from a patient, a memory circuit, and a controller circuit. The controller circuit can be configured to: determine baseline values of a signal feature from a first physiological signal sensed under a baseline condition of the patient; determine a feature value margin based on a variability metric of the baseline values of the signal feature; determine test values of the signal feature from a second physiological signal sensed under a test condition of the patient different from the baseline condition; and selectively store in the memory circuit a subset, less than an entirety, of the determined test values that fall outside the feature value margin about a reference feature value.

In Example 2, the subject matter of Example 1 optionally includes the baseline values and the test values of the signal feature each of which can be determined using windowed segments of respective the first or the second physiological signal, wherein the controller circuit is configured to store, in the memory circuit, time indices of windowed segments of the second physiological signal from which the stored test values are determined.

In Example 3, the subject matter of any one or more of Examples 1-2 optionally includes the variability metric that can include at least one of a range, a standard deviation, a variance, or a root-mean-squared (RMS) value of the baseline values of the signal feature.

In Example 4, the subject matter of any one or more of Examples 1-3 optionally includes the variability metric that can include a characteristic of a histogram of the baseline values of the signal feature.

In Example 5, the subject matter of any one or more of Examples 1-4 optionally includes the controller circuit that can be configured to determine the feature value margin using the variability metric scaled by an adjustable weight factor.

In Example 6, the subject matter of any one or more of Examples 1-5 optionally includes the controller circuit that can be configured to update the variability metric of the baseline values of the signal feature and to adjust the feature value margin in response to a user input via a user interface or a triggered event.

In Example 7, the subject matter of any one or more of Examples 1-6 optionally includes the controller circuit that can be configured to determine the reference feature value using a portion of the second physiological signal.

In Example 8, the subject matter of any one or more of Examples 1-7 optionally includes, wherein to selectively store the subset less than the entirety of the determined test values of the signal feature, the controller circuit is configured to: store in the memory circuit (i) the reference feature value and (ii) a first test value of the signal feature that falls outside the feature value margin about the reference feature value; update the reference feature value with the first test value; and store in the memory circuit a second test value of the signal feature that falls outside the feature value margin about the updated reference feature value.

In Example 9, the subject matter of any one or more of Examples 1-8 optionally include at least one of the first or the second physiological signal which can be an intrinsic physiological signal of the patient.

In Example 10, the subject matter of Example 9 optionally includes, wherein at least one of the first or the second physiological signal is an evoked response to electrostimulation of an anatomical target of the patient.

In Example 11, the subject matter of any one or more of Examples 1-10 optionally includes the signal feature that can include at least one of a signal amplitude range, a signal curve length representing accumulated signal amplitude differences over consecutive unit times, or a signal power.

In Example 12, the subject matter of any one or more of Examples 1-11 optionally includes an implantable medical device including at least the memory circuit, the controller circuit, and a communication circuit configured to transmit the stored test values of the signal feature to an external device.

In Example 13, the subject matter of Example 12 optionally includes the external device configured to interpolate signal feature values based on the stored test values of the signal feature and time indices of windowed segments of the second physiological signal from which the stored test values are determined.

In Example 14, the subject matter of Example 13 optionally includes, wherein to interpolate the signal feature values includes to use a linear interpolation or a nonlinear interpolation.

In Example 15, the subject matter of any one or more of Examples 1-14 optionally includes an electrostimulator configured to generate and deliver electrostimulation energy to an anatomical target of the patient based at least in part on the test values of the signal feature.

Example 16 is a method of managing physiological data collected in an ambulatory medical device. The method comprises steps of: determining baseline signal feature values from a first physiological signal sensed under a baseline condition of a patient; determining a feature value margin based on a variability metric of the baseline signal feature values; determining test signal feature values from a second physiological signal sensed under a test condition of the patient different from the baseline condition; and selectively storing in a memory circuit a subset, less than an entirety, of the determined test values that fall outside the feature value margin about a reference feature value.

In Example 17, the subject matter of Example 16 optionally includes the baseline signal feature values and the test signal feature values each of which can be determined using windowed segments of respective the first or the second physiological signal, the method further comprising storing, in the memory circuit, time indices of windowed segments of the second physiological signal from which the stored test values are determined.

In Example 18, the subject matter of any one or more of Examples 16-17 optionally includes determining the feature value margin using the variability metric scaled by an adjustable weight factor.

In Example 19, the subject matter of any one or more of Examples 16-18 optionally includes, in response to a user input via a user interface or a triggered event, updating the variability metric of the baseline values of the signal feature, and adjusting the feature value margin.

In Example 20, the subject matter of any one or more of Examples 16-19 optionally includes determining the reference feature value using a portion of the second physiological signal, wherein selectively storing the subset less than the entirety of the determined test values of the signal feature includes: storing in the memory circuit (i) the reference feature value and (ii) a first test value of the signal feature that falls outside the feature value margin about the reference feature value; updating the reference feature value with the first test value; and storing in the memory circuit a second test value of the signal feature that falls outside the feature value margin about the updated reference feature value.

In Example 21, the subject matter of any one or more of Examples 16-20 optionally includes establishing a communication link between (i) an implantable medical device comprising the memory circuit and (ii) an external device; and transmitting the stored test values of the signal feature to the external device via the communication link.

In Example 22, the subject matter of any one or more of Examples 16-21 optionally includes interpolating signal feature values based on the stored test signal feature values and time indices of windowed segments of the second physiological signal from which the stored test values are determined.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example, an example of an electrical therapy- delivery system.

FIG. 6 illustrates, by way of example and not limitation, a monitoring system and/or the electrical therapy-delivery system of FIG. 5, implemented using an implantable medical device (IMD).

FIG. 7 illustrates, by way of example and not limitation, a medical-device system with improved efficacy in physiological data storage and maintenance.

FIG. 8 illustrates an example of identifying a subset, less than an entirety, of signal feature values to be stored in an implantable device.

FIG. 9 is a flowchart illustrating an example method of storing and managing physiological data in an implantable device.

FIG. 10 is a flowchart illustrating an example method of selectively storing signal feature values in an implantable device based on a feature value margin determined under a baseline condition of the patient.

FIG. 11 illustrates generally a block diagram of an example machine upon which any one or more of the techniques (e.g., methodologies) discussed herein may perform.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments as described in this document implement efficient data storage and maintenance in battery-powered ambulatory medical devices, such as IMDs. Reduced data storage can be based on a signal feature value margin correlated to variability of baseline signal feature values determined from a physiological signal during a baseline condition of the patient. Various embodiments as described herein can lead to a reduction in memory access and data transmission, less physiological data to be maintained in an IMD, less stringent demand for device memory and battery capacity, and overall time and cost savings. Devices and methods described in this document may also allow users (e.g., healthcare professionals) to access device-stored historical data more efficiently, optimize individualized therapy and improve patient care.

This disclosure refers to evoked response (ER) signals acquired by implantable neuromodulation devices (such as ERNA signals acquired by implantable DBS device for treating neurological disorders such as Parkinson's Disease) as a nonlimiting example of physiological signals being analyzed, selectively stored, and/or transmitted to an external device. It is to be understood that the systems, devices, and methods as described in this document may also be used for analyzing, selecting storing, and/or transmitting other physiological signals or biomarker data that are either intrinsically generated or evoked by, for example, electrostimulation of an anatomical target. The electrostimulation may be therapeutic in nature in some examples, or diagnostic in nature in others.

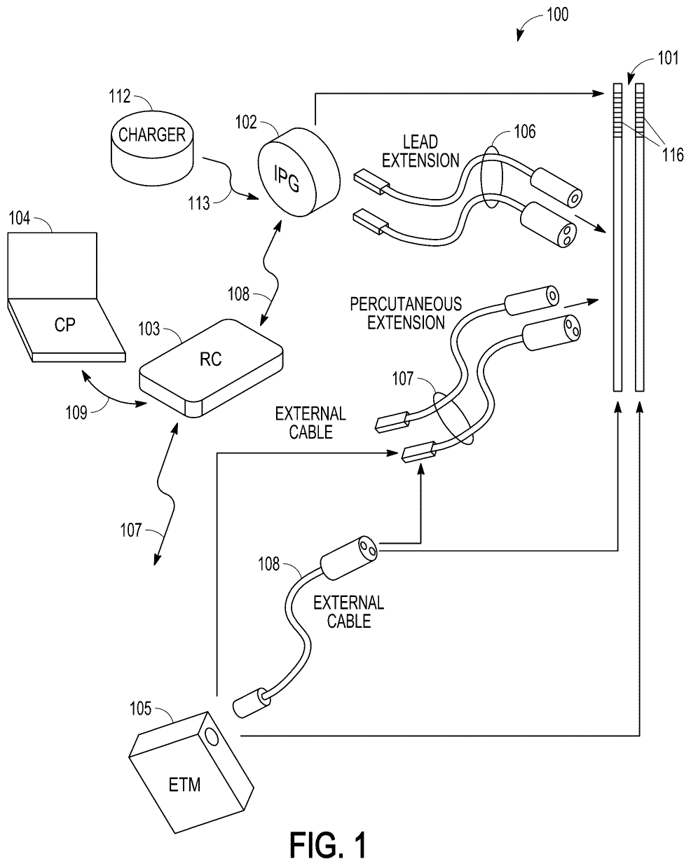

FIG. 1 illustrates, by way of example and not limitation, an electrical stimulation system 100, which may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

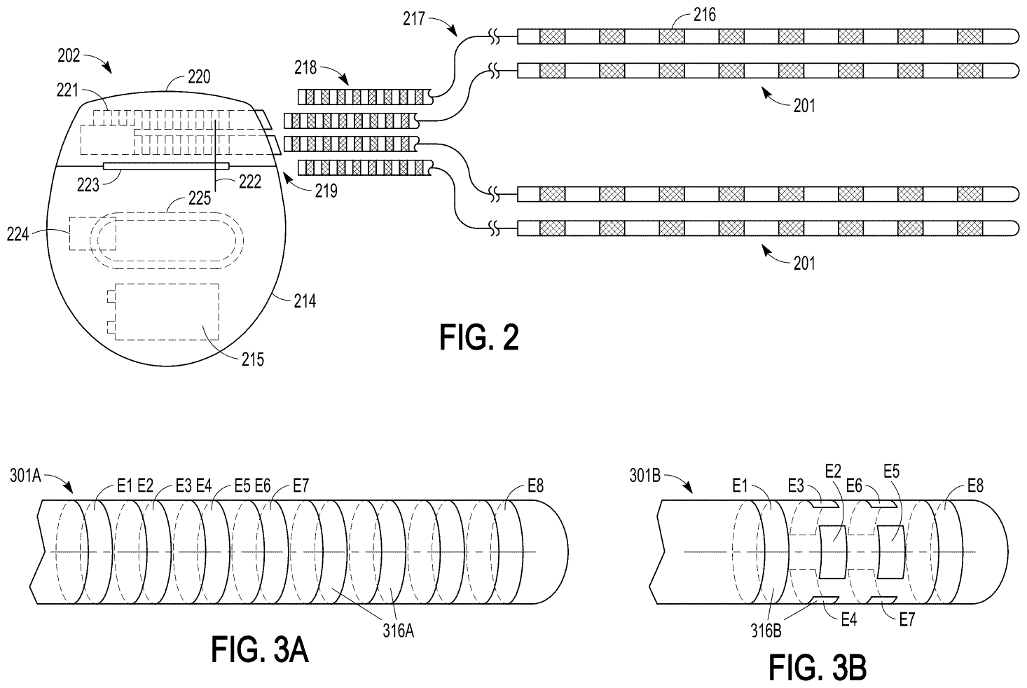

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more rows of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulating electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into rows of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all rows of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The rows of segmented electrodes may be positioned in irregular or regular intervals along a length of the lead 201.

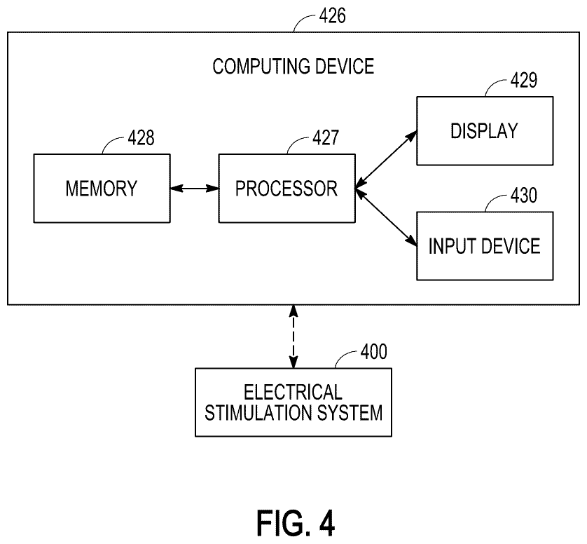

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1. The computing device may be used to perform process(s) for sensing parameter(s).

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, which is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

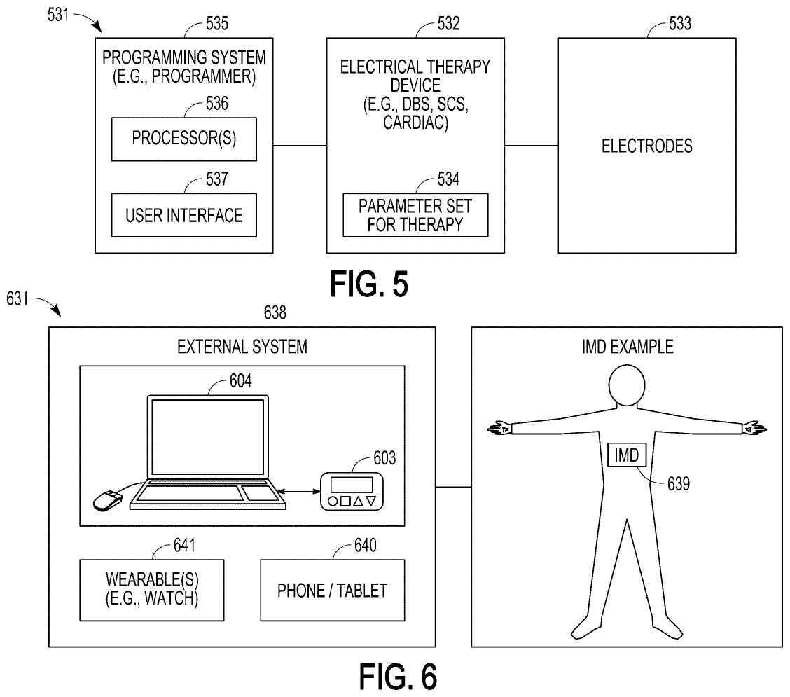

FIG. 5 illustrates, by way of example, an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, which may include one or more processors 536 and a user interface 537. The programming system 535 may be used to program and/or evaluate the parameter set(s) used to deliver the therapy. The illustrated system 531 may be a DBS system.

In some embodiments, the illustrated system 531 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 6 illustrates, by way of example and not limitation, a monitoring system and/or the electrical therapy-delivery system of FIG. 5, implemented using an implantable medical device (IMD). The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

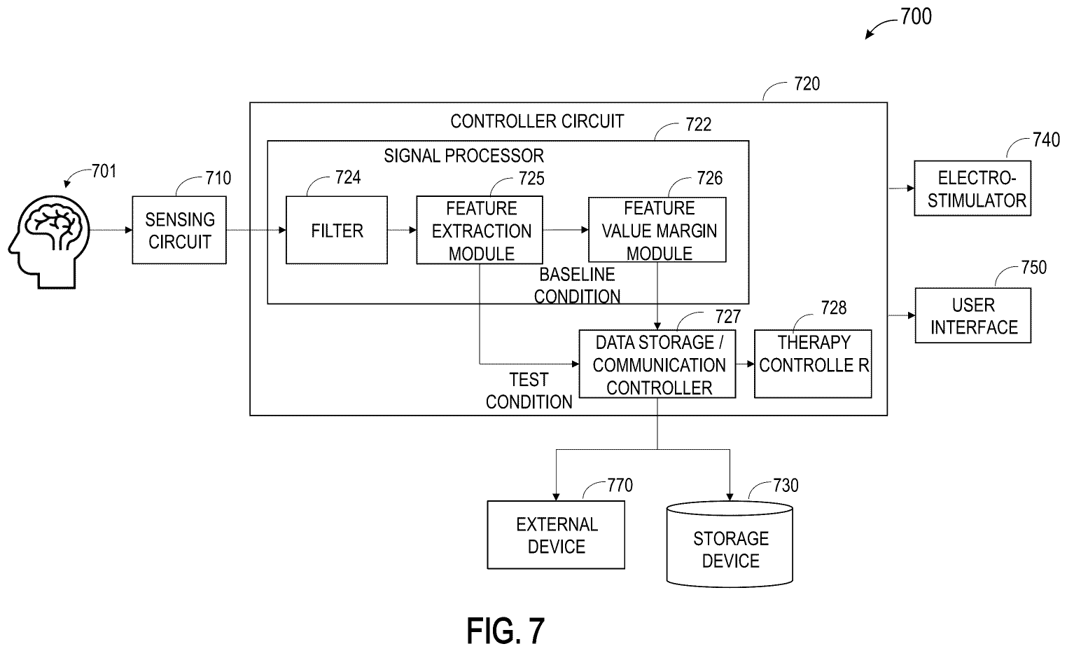

FIG. 7 illustrates, by way of example and not limitation, a medical-device system 700 with improved efficiency in physiological data storage and maintenance. The system 700 includes a sensing circuit 710, a controller circuit 720, a storage device 730, an electrostimulator 740, and a user interface 750. The system 700 may include an optional external device 760. Portions of the system 700 may be implemented in the IPG 102 or the CP 104.

The sensing circuit 710 may include or be associated with one or more sensors or sensing electrodes to sense physiological signals or biomarker data from a patient 701. Examples of the physiological signals or biomarker data may include electrocardiography (ECG), electromyography (EMG), and electroencephalography (EEG), electrospinogram (ESG), Evoked Resonant Neural Activity (ERNA) (also referred to as deep brain stimulation local evoked potentials (DLEP) or evoked oscillatory neural responses (EONR)), respiration, accelerometer in different locations (e.g., finger, spinal cord), transcutaneous impedance, temperature, and pupil dilation, among others. In an example, the sensing circuit 710 may be operatively connected to one or more leads and electrodes associated therewith, such as ring electrodes or segmented electrodes on the non-directional lead 301A or the directional lead 301B. The ring electrodes and/or the segmented electrodes may also be electrically coupled to the electrostimulator 740. The ring electrodes and/or the segmented electrodes may be configured as sensing electrodes for sensing ERs, or as stimulating electrodes for delivering electrostimulation pulses. The sensing circuit 710 may sense ERs from one or more sensing electrodes on a lead placed at a target site (e.g., a site of spinal cord or of brain) of the patient 701 in response to electrostimulation pulses delivered from a stimulating electrode at a stimulation site (e.g., a brain target). The ERs may be sensed in accordance with a stimulating-sensing electrode configuration. Commonly assigned U.S. Provisional Patent Application No. 63/529,959, entitled “EVOKED RESPONSE-GUIDED NEUROMODULATION LEAD PLACEMENT,” describes examples of stimulating-sensing electrode configuration that employs a full set, or a selected subset, of “off-diagonal” electrodes for ER sensing, the description of which is hereby incorporated by reference in its entirety.

The controller circuit 720 can include circuit sets comprising one or more other circuits or sub-circuits, such as a signal processor 722, a data storage/communication controller 727, and a therapy controller 728. The signal processor 722 may further include a filter 724, a feature extraction module 725, and a feature value margin module 726. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, one or more portions of the controller circuit 720 may be implemented as a part of a microprocessor circuit. The microprocessor circuit can be a dedicated processor such as a digital signal processor, application specific integrated circuit (ASIC), microprocessor, or other type of processor for processing information including physical activity information. Alternatively, the microprocessor circuit can be a general purpose processor that can receive and execute a set of instructions of performing the methods or techniques described herein.

The filter 724 can include a filter or a filter bank to filter the sensed physiological signal or biomarker data. In an example, a low-pass or band-pass filter, such as a moving average filter, may be used to filter out or attenuate high-frequency noise or interference from the received physiological signal or biomarker data. In some examples, filter parameters, such as corner frequencies and gains for the pass bands, can be adjustable or selectable such as from the user interface 750.

The feature extraction module 725 may extract one or more signal features from the filtered physiological signal or the filtered biomarker data. In an example of evoked response (ER) signal, ER features may include a signal amplitude, magnitude, peak value, value range, a signal curve length, a signal power or an RMS value within a time window (e.g., an epoch-averaged ER). The signal amplitude range or value range, also referred to as a peak-to-peak (P2P) value, can be measured as a difference between a maximum value or a minimum value of a dominant peak in the sensed evoked response or an epoch-averaged evoked response within the time window (also referred to as “max P2P” amplitude). Alternatively, the P2P value may be measured as a difference between a negative peak (trough) and an immediate subsequent positive peak (also referred to as “N1−P2 P2P” amplitude). The signal curve length can be measured as accumulated signal value differences of the sensed evoked response (or an epoch-averaged evoked response) over consecutive unit times (e.g., consecutive data sampling intervals) within the time window. The signal power can be measured as an area under the curve (AUC) of the sensed evoked response (or the epoch-averaged evoked response) within the time window.

In some examples, the feature extraction module 725 may extract signal features from each of a plurality of windowed segments of the filtered physiological signal. The windowed segments can be created by applying a sliding time window to the filtered physiological signal. The sliding time window has a window size or duration which can be programmable or adjustable. Temporally adjacent windowed segments may overlap at least in part in one example, or not overlap in another example. Signal feature values determined from respective windowed segments (e.g., AUC values of ER signal segments) are each associated with a time index of the corresponding time window. As such, the feature extraction module 725 essentially transforms the filtered physiological signal X(t) into a sequence (e.g., a time series) of signal feature values {F(1), F(2), . . . , F(n)} associated with time indices {t1, t2, . . . , tn}, where “n” represents the number of time windows for segmenting the physiological signal X(t).

In various examples, the sensing circuit 710 may sense physiological signals or biomarker data under different patient conditions. The feature extraction module 725 may accordingly generate signal feature values under different patient conditions. As illustrated in FIG. 7, baseline signal feature values {F_{b}(1), F_{b}(2), . . . , F_{b}(n)} may be determined from a physiological signal X_{b}(t) sensed under a baseline condition, such as when the patient is free of particular diseases or medical events. The feature value margin module 726 may determine a variability metric (σ) of the baseline signal feature values {F_{b}(1), F_{b}(2), . . . , F_{b}(n)}. Examples of the variability metric (σ) may include a range (between a maximum and a minimum), a standard deviation, a variance, or a root-mean-squared (RMS) value of the baseline signal feature values {F_{b}(1), F_{b}(2), . . . , F_{b}(n)}, among other variability measures. In some examples, the variability metric (σ) may be a characteristic of a histogram of the baseline signal feature values {F_{b}(1), F_{b}(2), . . . , F_{b}(n)}. The histogram represents an approximation of statistical distribution of the baseline signal feature values. An example characteristic of the histogram is a width of a histogram or distribution peak.

The feature value margin module 726 may determine a feature value margin (δ) based on the variability metric (σ) of the baseline signal feature values. In an example, the feature value margin may be defined as the variability metric σ scaled by a weight factor w: δ=w*σ. The weight factor w can take a value between 0 and 1. In an example, w is set to a fixed value (e.g., w=0.125). In some examples, the weight factor w can be adjusted by a user via the user interface 750, or automatically tuned in response to the a trigger event, such as a change in patient health status or a medical event.

A feature value range (also referred to as a threshold band) can be determined based on the feature value margin (δ) about a reference feature value F_{ref}. In an example, the feature value range is defined as (F_{ref}−δ, F_{ref}+δ). As will be discussed further below, the feature value range may be used as a selection criterion for identifying a subset, less than the entirety, of signal feature values obtained under different patient conditions to be stored in an onboard memory of an IMD. For example, signal feature values falling within the range (F_{ref}−δ, F_{ref}+δ) are deemed not substantially different than the reference feature value F_{ref}, and are “discarded” (i.e., not stored in the memory of the IMD nor transmitted to an external device) to save memory space and communication bandwidth and to reduce device power consumption.

Similar to the signal feature values determined during the baseline condition, the feature extraction module 725 may determine test signal feature values {F(1), F(2), . . . , F(n)} from a physiological signal X(t) sensed under a test condition of the patient different from the baseline condition. The data storage/communication controller 727 may identify a subset of test signal feature values, denoted by {(F*(1), F*(2), . . . , F*(m)}, less than the entirety of the test signal feature values {F(1), F(2), . . . , F(n)}, that fall outside the feature value margin (δ) (produced by the feature value margin module 726) about a reference feature value F_{ref}, where m (<n) represents the number of selected signal feature values from the original n test signal feature values {F(1), F(2), . . . , F(n)}. The identified subset {(F*(1), F*(2), . . . , F*(m)} can then be stored in the storage device 730, such as an onboard memory of the IPG 102 of FIG. 1 or the IMD 639 of FIG. 6.

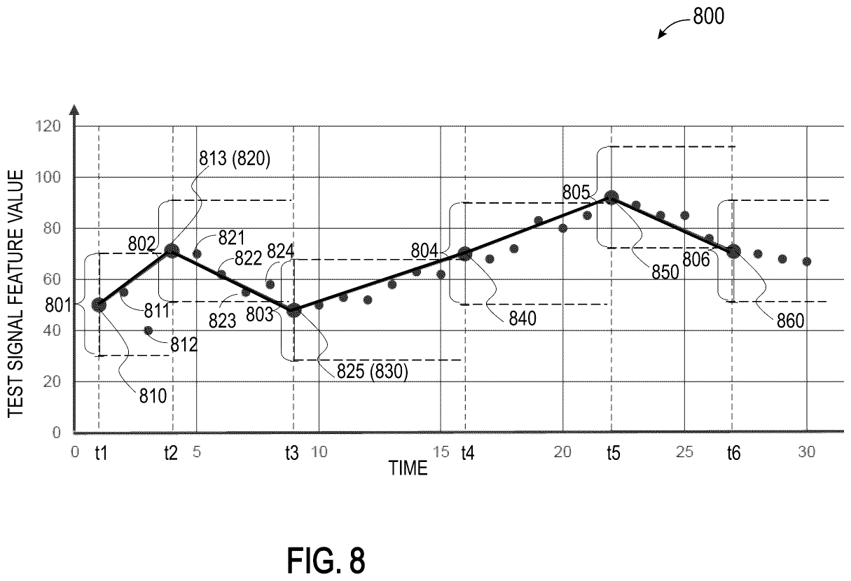

Referring now to FIG. 8, the diagram 800 illustrates an example of identifying a subset, less than an entirety, of signal feature values to be stored in a memory circuit of an implantable device. The signal feature values, plotted as filled circles, are determined from windowed segments of an ER signal acquired during a test condition of the patient. Each windowed segment is associated with a corresponding time index (in the x-axis). In this example, the feature value margin (δ), determined based on a variability metric of the baseline feature values, is 20 units. A reference value 810 can be determined using a portion (e.g., the first windowed segment) of the test physiological signal X(t). Alternatively, the reference value 810 may be determined based on the baseline values of the signal feature, such as an average of the baseline signal feature values {F_{b}(1), F_{b}(2), . . . , F_{b}(n)}.

The data storage/communication controller 727 may store the reference value 810 and the associated time index t1 in the storage device 730. The reference value 810, denoted by F_{ref}, can be used to define a feature value range 801 (F_{ref}−δ, F_{ref}+δ), where δ, as described above, represents a feature value margin correlated to a variability metric of the baseline signal feature values. The data storage/communication controller 727 can determine whether or not to store a subsequent feature value based on a comparison to the feature value range 801. In the instant example, because signal feature values 811 and 812 are both within the value range 801, they are not stored in the storage device 730. The next feature value 813 is outside the range 801, and therefore is stored in the storage device 730.

The data storage/communication controller 727 may then update the reference value F_{ref} with the feature value 813 (also referenced as 820), and redefine a new feature value range 802 with respect to the updated F_{ref} 820 (with the same margin δ). Subsequent signal feature values 821, 822, 823, and 824 are all within the value range 802, and are not stored in the storage device 730. Signal feature value 825 is outside the value range 802, therefore gets stored in the storage device 730. The data storage/communication controller 727 may then update the reference value F_{ref} with the latest stored feature value 825 (also referenced as 830), and redefine a new feature value range 803 with respect to the updated F_{ref} 830 (with the same margin δ).

The above process may continue until all the test signal feature values {F(1), F(2), . . . , F(n)} have been analyzed, with each signal feature value either being discarded or stored in the storage device 730. In the illustrated example, instead of storing all the test signal feature values, only a selected subset including feature values 820, 830, 840, 850, and 860, along with their respective time indices t2, t3, t4, t5, and t6, are stored in the storage device 730 because they each falls outside their respective feature value ranges 801, 802, 803, 804, and 805.

Referring back to FIG. 7, in an example, the controller circuit 720 and the storage device 730 may be implemented in an implantable medical device (such as the IPG 102 of FIG. 1 or the IMD 639 of FIG. 6) operatively in communication with an external device 760 (e.g., a physician's programmer, a remote data server, or a mobile device). The signal feature values selectively stored in the implantable device may be transmitted (or “downloaded”) to the external device 760. The transmission may be initiated in a command mode or an event-triggered mode, periodically, or accordingly to a set schedule (e.g., once every day at a set time). The selective data storage and/or data transmission as described requires fewer memory accesses and less data transmission, reduces the amount of physiological data to be maintained in the implantable device, and reduces the demand for device memory and battery capacity. It also allows a user to access device-stored historical data with reduced cost and higher efficiency.

The selectively stored feature values {(F*(1), F*(2), . . . , F*(m)}, such as feature values 820, 830, 840, 850, and 860 as illustrated in FIG. 8, may be unevenly or irregularly spaced in time. To facilitate data inspection and/or further processing, in various examples, the external device 760 may “reconstruct” a substantially evenly-spaced time series of feature values through an interpolation process of the stored signal feature values {(F*(1), F*(2), . . . , F*(m)}. The interpolation can be a linear, a piecewise linear, or a nonlinear (e.g., splines) interpolation based on both the downloaded signal feature values and their corresponding time indices.

In the example with respect to FIG. 8, the data storage/communication controller 727 updates the feature value range (F_{ref}−δ, F_{ref}+δ) by updating the reference feature value F_{ref}, while keeping the margin δ constant during the feature value selection process. The margin δ may affect the number of feature values (i.e., the number “m”) to be stored in the storage device. Generally, a smaller margin δ corresponds to a narrower value range (F_{ref}−δ, F_{ref}+δ), such that more feature values (i.e., a larger “m”) would likely fall outside said range and therefore get stored in the storage device. Conversely, a larger margin δ corresponds to a wider value range (F_{ref}−δ, F_{ref}+δ), such that fewer feature values (i.e., a smaller “m”) would likely fall outside said range and get stored or in the storage device. In some examples, the feature value margin module 726 may adjust the margin δ to control the number of feature values to be stored (or discarded). In an example where δ is determined using δ=w*σ, the feature value margin module 726 may update the margin δ by adjusting at least one of the variability metric σ of the baseline signal feature values, or the weight factor w. The weight factor w and/or the variability metric σ may be adjusted by a user via the user interface 750, or in response to a triggered event. For example, when a medical event or a change in patient health condition warrants more signal feature values to be stored and reviewed by a clinician, the weight factor w may be manually or automatically decreased to produce a smaller margin δ, hence a narrower value range that would cause more feature values to be selected and stored and downloaded to the external device 770 for user review or for further processing. In some examples, the selectively stored feature values may be evaluated against a data reduction performance criterion, such as a difference between the original test signal feature values {F(1), F(2), . . . , F(n)} and the “reconstructed” feature value time series. If said difference is outside an acceptable range (indicating too “harsh” the data reduction), then the weight factor w may be decreased to produce a smaller margin δ, hence a narrower value range that would cause more feature values to be selected, stored, and transmitted.

As stated above, the data storage/communication controller 727 can store a subset of m feature values {(F*(1), F*(2), . . . , F*(m)} selected from the entire set of n feature values {F(1), F(2), . . . , F(n)}. Alternatively, in some examples, the data storage/communication controller 727 may store the entire n feature values, such as when the storage device 730 has sufficient storage space. Once the storage is full, at least a portion of the stored data may be overwritten with newly selected feature values. In some examples, in addition or alternative to storing feature values, raw physiological signal or biomarker data produced by the sensing circuit 720, and/or processed data (such as filtered signal data produced by the filter 724), may be stored in the storage device 730. In response to a user command or a trigger event, signal data or feature values stored in the storage device 730 can be transmitted to the external device 770. The amount of data to be transmitted, or the time or manner of transmission, may be adjustable or selectable by the user.

The therapy controller 728 can generate a control signal to the electrostimulator 740 to adjust the neuromodulation therapy based on the selected feature values that are stored in the storage device 730. The electrostimulator 740 may be configured to deliver electrical stimulation according to a stimulation setting. The electrical stimulation may be delivered using a monopolar (far-field) or a bipolar (near-field) configuration. Examples of the therapy setting may include, electrode selection and configuration, stimulation parameter values including, for example, amplitudes, pulse width, frequency, pulse waveform, active or passive recharge mode, ON time, OFF time, therapy duration, and fractionalization, among others. In an example, the therapy controller 728 can be implemented as a proportional integral (PI) controller, a proportional-integral-derivative (PID) controller, or other suitable controller that takes the comparison of the sensed physiological signals or the selected feature values to an acceptance criterion (e.g., an ER template) as a feedback on the adjustment of stimulation settings. The types of data, and the recordings used to produce them, may vary regarding the type of acceptance criteria and operations employed. For example, ER data used to drive decisions about the electrode selection and configuration may differ from data and evoke/record configurations used to compare to acceptance criteria and use as control signal for amplitude adjustment. One ER measurement may be used to inform lead positioning (e.g., by sweeping a non-therapeutic sampling pulse across the space of the lead electrodes), another ER measurement may be used to determine or adjust a stimulation parameter (e.g., by sweeping a therapeutic sampling pulse across amplitudes).

The electrostimulator 740 can be an implantable module, such as incorporated within the IPG 10. Alternatively, the electrostimulator 740 can be an external stimulation device, such as incorporated with the ETS 40. In some examples, the user can choose to either send a notification (e.g., to the RC 45 or a smartphone with the patient) for a therapy reminder, or to automatically initiate or adjust neuromodulation therapy in accordance with the adjusted therapy setting. If an automatic therapy initiation is selected, the electrostimulator 740 can deliver stimulation in accordance with the adjusted therapy setting.

In some examples, the therapy controller 728 can generate a recommendation to the user to reposition the lead or to adjust the device setting (e.g., a programmable parameter of the electrostimulator 740). The repositioning of the lead or the adjustment of the device setting can cause the sensed ERs to align or more favorably compare to the acceptance criteria (e.g., an ER template) during an implantation procedure. In some examples, the therapy controller 728 may determine or modify therapeutic stimulation settings based on the sense ERs or features or a distribution of the features thereof. The electrostimulator 740 may deliver therapeutic stimulation (e.g., DBS) in accordance with the determined or modified therapeutic stimulation settings.

In some embodiments the display may provide a suggestion to the user to adjust stimulation parameters to cause the since developed responses to more favorably compare to the acceptance criteria (e.g., an ER template). The recommendation can be displayed on the user interface 750. The user interface 750 can be a portable (e.g., handheld) device, such as the RC 45 or a smartphone (with executable software application) operable by the patient at his or her home without requiring extra clinic visits or consultation with a device expert. In another example, the user interface 750 can be a programmer device, such as the CP 50. In addition to the recommendation for lead replacement, other information may be displayed on the user interface 750 including, by way of example and not limitation, one or more of the sensed ERs (including, for example, before and/or after filtering), ER features, distribution of ER features, the acceptance criteria (e.g., one or more ER templates), or the comparison between the sensed ERs and the acceptance criteria.

In some examples, the user interface 750 allows a physician to remotely review therapy settings and treatment history, consult with the patient to obtain information including pain relief and SCS-related side effects or symptoms, perform remote programming of the electrostimulator 740, or provide other treatment options to the patient. The user interface 750 can allow a user (e.g., the patient, the physician managing the patient, or a device expert) to view, program, or modify a device setting. For example, the user may use one or more user interface (UI) control elements to provide or adjust values of one or more device parameters, or select from a plurality of pre-defined stimulation programs for future use. Each stimulation program can include a set of stimulation parameters with respective pre-determined values. In some examples, the user interface 750 can include a display to display textually or graphically information provided by the user via an input unit, and device settings including, for example, feature selection, sensing configurations, signal pre-processing settings, therapy settings, optionally with any intermediate calculations. In an example, the user interface 750 may present to the user an “optimal” or improved therapy setting, such as determined based on a closed-loop or adaptive feedback control of electrostimulation based on a selected evoked response signal feature, in accordance with various embodiments discussed in this document. In some examples, the user can use the user interface 750 to provide feedback on a neuromodulation therapy, including, for example, side effects or symptoms arise or persist associated with the neurostimulation, or severity of the symptom or a side effect.

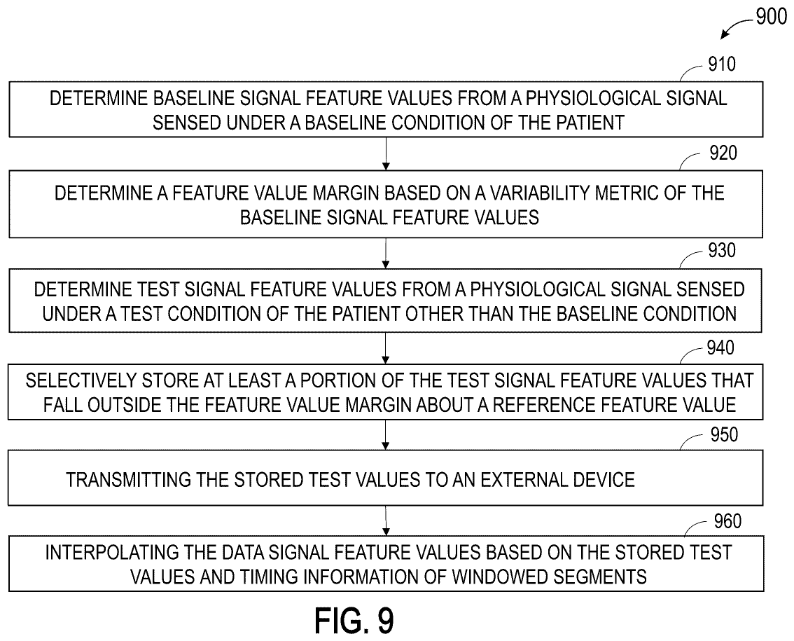

FIG. 9 is a flowchart illustrating an example method 900 of storing and managing physiological data in an implantable device. The method 900 may be implemented in and executed by a medical system such as the medical-device system 700. In an example, the method 900 may be implemented in a programmer device such as RC 45 or CP 50 in communication with an electrostimulator such as IPG 10 or electrostimulator 740.

At step 910, baseline signal feature values can be determined from a physiological signal sensed under a baseline condition of the patient, such as when the patient is free of particular diseases or medical events. Examples of the physiological signal or biomarker data may include electrocardiography (ECG), electromyography (EMG), and electroencephalography (EEG), electrospinogram (ESG), Evoked Resonant Neural Activity (ERNA), respiration, accelerometer in different locations (e.g., finger, spinal cord), transcutaneous impedance, temperature, and pupil dilation, among others. The physiological signal may be filtered, and one or more signal features can be extracted from the filtered physiological signal. The signals features may include, for example, signal amplitude, magnitude, peak value, value range, a signal curve length, a signal power or an RMS value. In an example, the baseline signal feature values can each be determined from windowed segments of the physiological signal sensed under the baseline condition. Temporally adjacent windowed segments may overlap at least in part in one example, or not overlap in another. Each baseline signal feature value can be associated with a time index of the corresponding time window.

At step 920, a feature value margin can be determined based on a variability metric of the baseline signal feature values, such as using the feature value margin module 726. As described above with respect to FIG. 7, the feature value margin δ may be defined as δ=w*σ, where σ represents a variability metric of the baseline signal feature values, and w represents a weight factor taking a value between 0 and 1. Examples of the variability metric σ include a range, a standard deviation, a variance, or a root-mean-squared (RMS) value of the baseline values of the signal feature, or a characteristic of a histogram of the baseline values of the signal feature. The variability metric σ and/or the weight factor w can be adjusted in response to a user input via a user interface, or automatically tuned in response to the a trigger event such as a change in patient health status or a medical event.

At step 930, test signal feature values can be determined from a physiological signal sensed under a test condition of the patient different from the baseline condition. Similar to the baseline signal feature values determined during the baseline condition, the test signal feature values can each be determined from windowed segments of the physiological signal sensed under the test condition. Each test signal feature value can be associated with a time index of the corresponding time window.

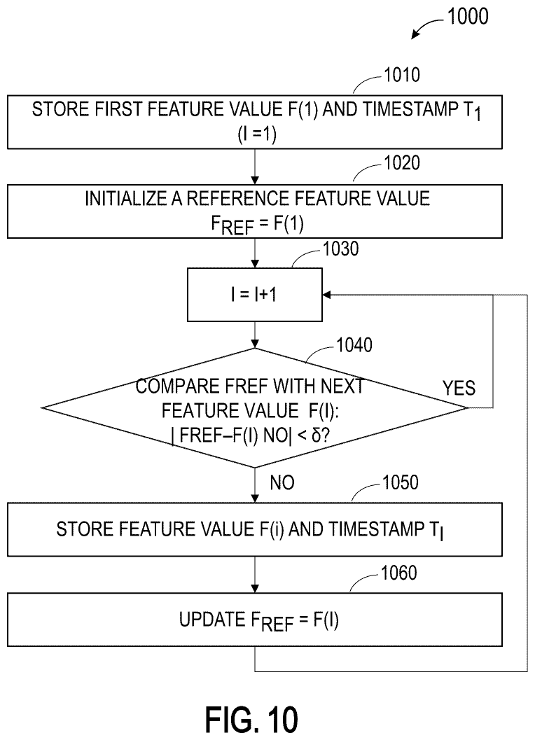

At step 940, a subset, less than the entirety, of the test signal feature values that fall outside the feature value margin about a reference feature value can be identified, and stored in a memory circuit of an implantable medical device, such as using the data storage/communication controller 727. As described above with respect to FIG. 7, test signal feature values falling within a range (F_{ref}−δ, F_{ref}+δ) defined by the feature value margin (δ) about the reference feature value (F_{ref}) are deemed to be no substantially different than the reference feature value F_{ref}, and are “discarded” (i.e., not stored in an onboard memory of the implantable device and/or transmitted to an external device) to save memory space and communication bandwidth and reduce power consumption. An example method of selective storage of signal feature values based on the feature value margin about a reference feature value are described below with respect to FIG. 10.

At step 950, the selectively stored test signal feature values may be transmitted to an external device 760 via a communication link, such as a wireless communication link established between an implantable medical device comprising the memory circuit and the external device.

At step 960, a substantially evenly-spaced time series of feature values may be reconstructed through an interpolation process of the stored signal feature values. The reconstruction process, which may be carried out in the external device, may involve interpolating signal feature values based on the selectively stored and/or transmitted test signal feature values and the corresponding time indices of the windowed segments of the physiological signal. The interpolation can be a linear, a piecewise linear, or a nonlinear (e.g., splines) interpolation based on both the downloaded signal feature values and their corresponding time indices. The interpolated signal feature values may facilitate data inspection and/or further processing.

FIG. 10 is a flowchart illustrating an example method 1000 of selectively storing signal feature values in an implantable device based on a feature value margin determined under a baseline condition of the patient. The method 1000 can be an example of step 940 of the method 900.

At step 1010, a first test signal feature value F(1), determined from a windowed segment of a physiological signal sensed under a test condition of the patient, is stored in the device memory. The time index of the windowed segment for determining F(1) can also be stored in the device memory.

At step 1020, a reference feature value F_{ref} can be initialized to F(1). At step 1030, the windowed segment index moves to the next, and the signal feature value (e.g., F(2)) can be determined from that window segment. At step 1040, the feature value F(2) is compared to the reference value F_{ref}, which is then set to F(1). If F(2) is within the margin δ (as determined at step 920 of method 900) about the reference value F_{ref} (that is, if F(2) is within the range (F_{ref}−δ, F_{ref}+δ)), then F(2) is deemed no substantially different from F_{ref} and is not stored. The process then goes on at step 1030 to check the next feature value F(3). If at step 1040 the feature value (e.g., F(2)) falls outside the margin δ about the reference value F_{ref} (that is, F(2) is outside the range (F_{ref}−δ, F_{ref}+δ)), then F(2) is deemed substantially different from F_{ref}, and is stored in the memory at step 1050.

At step 1060, the reference value F_{ref} may be updated with the stored feature value F(2). The process then continues at step 1030 to check the next feature value, until all the feature values are analyzed or a stop condition is met. The stored feature values, along with their respective time indices, can be transmitted to an external device, as described above with respect to FIG. 9, step 950.

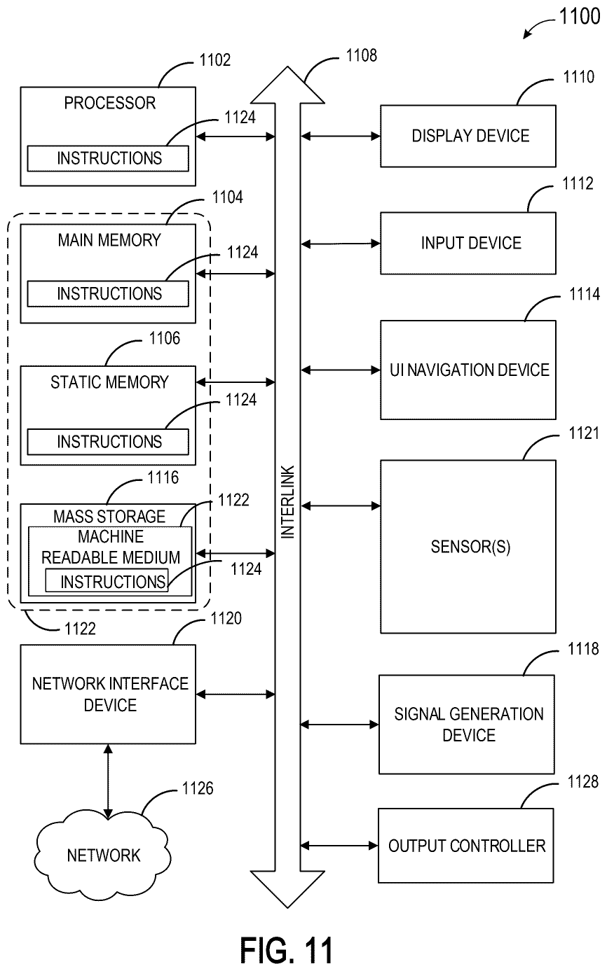

FIG. 11 illustrates generally a block diagram of an example machine 1100 upon which any one or more of the techniques (e.g., methodologies) discussed herein may perform. Portions of this description may apply to the computing framework of various portions of the medical device or the external programmer device as described above.

In alternative examples, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), among other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 1100 may include a hardware processor 1102 (e.g., a central processing unit (CPU), a graphics processing unit (GPU), a hardware processor core, algorithm specific ASIC, or any combination thereof), a main memory 1104 and a static memory 1106, some or all of which may communicate with each other via an interlink (e.g., bus) 1108. The machine 1100 may further include a display unit 1110 (e.g., a raster display, vector display, holographic display, etc.), an alphanumeric input device 1112 (e.g., a keyboard), and a user interface (UI) navigation device 1114 (e.g., a mouse). In an example, the display unit 1110, input device 1112 and UI navigation device 1114 may be a touch screen display. The machine 1100 may additionally include a storage device (e.g., drive unit) 1116, a signal generation device 1118 (e.g., a speaker), a network interface device 1120, and one or more sensors 1121, such as a global positioning system (GPS) sensor, compass, accelerometer, or other sensors. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1116 may include a machine-readable medium 1122 on which is stored one or more sets of data structures or instructions 1124 (e.g., software) embodying or utilized by any one or more of the techniques or functions described herein. The instructions 1124 may also reside, completely or at least partially, within the main memory 1104, within static memory 1106, or within the hardware processor 1102 during execution thereof by the machine 1100. In an example, one or any combination of the hardware processor 1102, the main memory 1104, the static memory 1106, or the storage device 1116 may constitute machine readable media.

While the machine-readable medium 1122 is illustrated as a single medium, the term “machine readable medium” may include a single medium or multiple media (e.g., a centralized or distributed database, and/or associated caches and servers) configured to store the one or more instructions 1124.

The term “machine readable medium” may include any medium that is capable of storing, encoding, or carrying instructions for execution by the machine 1100 and that cause the machine 1100 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 1124 may further be transmitted or received over a communication network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 1120 may include one or more physical jacks (e.g., Ethernet, coaxial, or phone jacks) or one or more antennas to connect to the communication network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1100, and includes digital or analog communications signals or other intangible medium to facilitate communication of such software.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. An ambulatory medical-device system, comprising:
a sensor circuit configured to sense a physiological signal from a patient;
a memory circuit; and
a controller circuit configured to:
determine baseline values of a signal feature from a first physiological signal sensed under a baseline condition of the patient;
determine a feature value margin based on a variability metric of the baseline values of the signal feature;
determine test values of the signal feature from a second physiological signal sensed under a test condition of the patient different from the baseline condition; and
selectively store in the memory circuit a subset, less than an entirety, of the determined test values that fall outside the feature value margin about a reference feature value.

2. The ambulatory medical-device system of claim 1, wherein the baseline values and the test values of the signal feature are each determined using windowed segments of respective the first or the second physiological signal,
wherein the controller circuit is configured to store, in the memory circuit, time indices of windowed segments of the second physiological signal from which the stored test values are determined.

3. The ambulatory medical-device system of claim 1, wherein the variability metric includes at least one of a range, a standard deviation, a variance, or a root-mean-squared (RMS) value of the baseline values of the signal feature.

4. The ambulatory medical-device system of claim 1, wherein the variability metric include a characteristic of a histogram of the baseline values of the signal feature.

5. The ambulatory medical-device system of claim 1, wherein the controller circuit is configured to determine the feature value margin using the variability metric scaled by an adjustable weight factor.

6. The ambulatory medical-device system of claim 1, wherein the controller circuit is configured to, in response to a user input via a user interface or a triggered event, update the variability metric of the baseline values of the signal feature and adjust the feature value margin.

7. The ambulatory medical-device system of claim 1, wherein the controller circuit is configured to determine the reference feature value using a portion of the second physiological signal.

8. The ambulatory medical-device system of claim 1, wherein to selectively store the subset less than the entirety of the determined test values of the signal feature, the controller circuit is configured to:
store in the memory circuit (i) the reference feature value and (ii) a first test value of the signal feature that falls outside the feature value margin about the reference feature value;
update the reference feature value with the first test value; and
store in the memory circuit a second test value of the signal feature that falls outside the feature value margin about the updated reference feature value.

9. The ambulatory medical-device system of claim 1, wherein at least one of the first or the second physiological signal is an evoked response to electrostimulation of an anatomical target of the patient.

10. The ambulatory medical-device system of claim 1, wherein the signal feature includes at least one of a signal amplitude range, a signal curve length representing accumulated signal amplitude differences over consecutive unit times, or a signal power.

11. The ambulatory medical-device system of claim 1, comprising an implantable medical device that includes at least the memory circuit, the controller circuit, and a communication circuit configured to transmit the stored test values of the signal feature to an external device.

12. The ambulatory medical-device system of claim 11, further comprising the external device configured to interpolate signal feature values based on the stored test values of the signal feature and time indices of windowed segments of the second physiological signal from which the stored test values are determined.

13. The ambulatory medical-device system of claim 1, comprising an electrostimulator configured to generate and deliver electrostimulation energy to an anatomical target of the patient based at least in part on the test values of the signal feature.

14. A method of managing physiological data collected in an ambulatory medical device, the method comprising:
determining baseline signal feature values from a first physiological signal sensed under a baseline condition of a patient;
determining a feature value margin based on a variability metric of the baseline signal feature values;
determining test signal feature values from a second physiological signal sensed under a test condition of the patient different from the baseline condition; and
selectively storing in a memory circuit a subset, less than an entirety, of the determined test values that fall outside the feature value margin about a reference feature value.

15. The method of claim 14, wherein the baseline signal feature values and the test signal feature values are each determined using windowed segments of respective the first or the second physiological signal, the method further comprising storing, in the memory circuit, time indices of windowed segments of the second physiological signal from which the stored test values are determined.

16. The method of claim 14, wherein determining the feature value margin includes using the variability metric scaled by an adjustable weight factor.

17. The method of claim 14, further comprising, in response to a user input via a user interface or a triggered event, updating the variability metric of the baseline values of the signal feature, and adjusting the feature value margin.

18. The method of claim 14, further comprising determining the reference feature value using a portion of the second physiological signal,
wherein selectively storing the subset less than the entirety of the determined test values of the signal feature includes:
storing in the memory circuit (i) the reference feature value and (ii) a first test value of the signal feature that falls outside the feature value margin about the reference feature value;
updating the reference feature value with the first test value; and
storing in the memory circuit a second test value of the signal feature that falls outside the feature value margin about the updated reference feature value.

19. The method of claim 14, further comprising:
establishing a communication link between (i) an implantable medical device comprising the memory circuit and (ii) an external device; and
transmitting the stored test values of the signal feature to the external device via the communication link.

20. The method of claim 14, further comprising interpolating signal feature values based on the stored test signal feature values and time indices of windowed segments of the second physiological signal from which the stored test values are determined.
